(19) United States
(12) Patent Application Publication
(10) Pub. No.: US 2026/0041877 A1
(43) Pub. Date: Feb. 12, 2026

(54) FORM FITTING TERMINALLY STERILIZABLE PACKAGING SYSTEMS FOR CATHETERS

(71) Applicant: EDWARDS LIFESCIENCES CORPORATION, Irvine, CA (US)

(72) Inventors: Amrut Nandan Biswal, Irvine, CA (US); Arti Prasad Roth, Lake Forest, CA (US); Salih Yildiz, Irvine, CA (US)

(21) Appl. No.: 19/293,867

(22) Filed: Aug. 7, 2025

Related U.S. Application Data
(60) Provisional application No. 63/680,749, filed on Aug. 8, 2024.

(51) Int. Cl. A61M 25/00; B65D 81/05

(52) U.S. Cl. CPC A61M 25/002; B65D 81/052

(57) ABSTRACT

In one aspect, a form-fitting packaging container for a catheter includes a hollow body and a plurality of supports disposed in the hollow body. The plurality of supports define a central opening configured to receive the catheter. The central opening is concentric with the hollow body. In another aspect, method of assembling a form-fitting packaging container for a catheter includes providing a hollow body, providing a plurality of supports in the hollow body, where each support defines a central opening; and inserting a catheter through the central openings of the plurality of supports.

### RELATED APPLICATIONS

This application claims the benefit of Provisional Application No. 63/680,749, filed on Aug. 8, 2024, which is hereby incorporated by reference in its entirety.

### FIELD OF INVENTION

The present disclosure is generally directed to packaging systems and more particularly to packaging terminally sterilized medical devices.

### BACKGROUND

Current technology for packaging terminally sterilized medical devices includes a combination of backer cards, trays, and Tyvek®-backed pouches. Current packaging containers include significant wasted space, which increases the space required for shipping and storing the packaged medical devices. This is particularly true for long catheters, which must be retained in a packaging container in a fully extended state.

A need exists for packaging containers that reduce wasted packaging volume while protecting the medical device from damage during shipping and long-term storage.

### SUMMARY

The present disclosure relates to solving the issues identified above with numerous form-fitting packaging containers and systems that more closely conform to the shape of the packaged device while supporting and protecting the packaged device during transport and storage.

In one aspect, a form-fitting packaging container for a catheter includes a hollow body extending longitudinally between a first and a second end and a plurality of supports disposed in the hollow body and spaced longitudinally between the first end and the second end. The plurality of supports define a central opening configured to receive the catheter.

In another aspect, method of assembling a form-fitting packaging container for a catheter includes providing a hollow body extending longitudinally, providing a plurality of supports in the hollow body, where each support defines a central opening; and inserting a catheter through the central openings of the plurality of supports.

The present summary is provided only by way of example, and not limitation. Other aspects of the present disclosure will be appreciated in view of the entirety of the present disclosure, including the entire text, claims and accompanying figures.

### BRIEF DESCRIPTION OF THE DRAWINGS

FIG. 1 is a schematic view of one example of a catheter.

FIG. 2 is a schematic illustration of one example of a form-fitting packaging container configured for transport and storage of the catheter of FIG. 1.

FIG. 3 is a schematic illustration of one example of a support attached to an exoskeleton of the form-fitting packaging container of FIG. 2.

FIG. 4 is a schematic illustration of another example of a form-fitting packaging container configured for transport and storage of the catheter of FIG. 1.

FIG. 5 is a schematic illustration of one example of a support attached to an exoskeleton of the form-fitting packaging container of FIG. 4.

FIG. 6 is a schematic illustration of another example of a support attached to an exoskeleton of the form-fitting packaging container of FIG. 4.

FIG. 7 is a schematic illustration of yet another example of a form-fitting packaging container configured for transport and storage of the catheter of FIG. 1.

FIG. 8 is a schematic illustration of yet another example of a form-fitting packaging container configured for transport and storage of the catheter of FIG. 1.

FIG. 9 is a schematic illustration of one example of an actuating support attached to a body of the form-fitting packaging container of FIG. 8.

FIG. 10 is a schematic illustration of another example of an actuating support attached to a body of the form-fitting packaging container of FIG. 8.

FIG. 11 is a schematic illustration of yet another example of a form-fitting packaging container configured for transport and storage of the catheter of FIG. 1.

FIG. 12 is a schematic cross-sectional view of a section of the form-fitting packaging container taken along the 12-12 line of FIG. 11.

While the above-identified figures set forth embodiments of the present invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of the principles of the invention. The figures may not be drawn to scale, and applications and embodiments of the present invention may include features, steps and/or components not specifically shown in the drawings.

### DETAILED DESCRIPTION

The disclosed form-fitting packaging containers and systems disclosed herein are configured to provide stability of a medical device during shipping and in long-term storage. The disclosed packaging systems are specifically suited for catheter packing, but it will be understood by one of ordinary skill in the art that other devices (medical or non-medical) of similar shape and/or size can be packaged using the form-fitting packaging containers and systems disclosed.

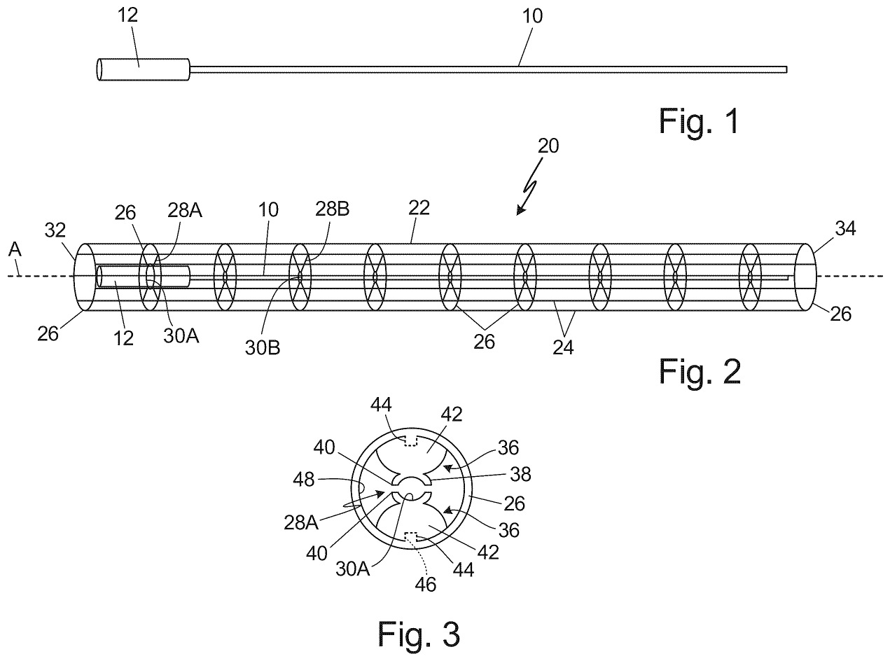

FIG. 1 is a schematic view of a catheter. FIG. 1 shows catheter 10 having a handle 12. Catheter 10 can be a venous catheter, extending in length, for example, up to 65 inches. Catheter 10 is packaged for transport and storage in the extended orientation, as required for medical use. Coiling or otherwise bending catheter 10 for transport and storage can alter the mechanical shape of catheter 10, which can be problematic when catheter 10 is used in a medical procedure. Handle 12 can have a variety of shapes and sizes. As disclosed herein, the diameter or cross-sectional area of handle 12 can in part or wholly define the size of the packaging container used for transport and storage of catheter 10. The form-fitting packaging containers disclosed herein are designed for universal use with a variety of catheter sizes and handle configurations. The form-fitting packaging containers are configured to contain the entirety of catheter 10, including handle 12. Each of the form-fitting packaging containers described herein have a hollow body that is cylindrical to generally conform to the shape of catheter 10 and handle 12. It will be understood by one of ordinary skill in the art that the shape of any of the disclosed form-fitting packaging containers described herein can be modified to more closely conform to devices of differing shapes and are not limited to the cylindrical configurations shown.

FIG. 2 is a schematic illustration of one example of a form-fitting packaging container configured for transport and storage of catheter 10. FIG. 2 shows form-fitting packaging container 20, exoskeleton 22, rails 24, rings 26, supports 28A and 28B, central openings 30A and 30B, first end 32, second end 34, longitudinal axis A, and catheter 10 with handle 12. Catheter 10 with handle 12 is disposed in form-fitting packaging container 20.

Form-fitting packaging container 20 has exoskeleton 22 formed by a plurality of rails 24 and a plurality of rings 26. Exoskeleton 22 is a cylindrical hollow body having a cage configuration, with openings between adjacent rails 24 and adjacent rings 26 (lead lines identifying just two of the plurality of rails 24 and five of the plurality of rings 26 shown in the form-fitting packaging container 20). Rails 24 extend longitudinally between first end 32 and second end 34, opposite first end 32. First and second ends 32 and 34 are terminal ends of exoskeleton 22. Rails 24 define the length of exoskeleton 22. The length of exoskeleton 22 can be selected to provide full containment of catheter 10 with handle 12 in a fully extended orientation. Rails 24 extend straight from first end 32 to second end 34 and parallel to each other.

In a similar manner, rings 26 define the shape and diameter of exoskeleton 22. In some embodiments, rings 26 have a diameter greater than the largest diameter of catheter 10 (i.e., greater than the diameter of handle 12) to help isolate catheter 10 from external stresses applied to exoskeleton 22. Each ring 26 is connected to each of the plurality of all rails 24 (e.g., all the rails). Rails 24 can be spaced equidistantly about a circumference of each ring 26. Adjacent rings 26 are interconnected by rails 24. Openings of rings 26 are sized to receive catheter 10 with handle 12. In some embodiments, a first ring 26 is disposed at first end 32, a second ring 26 is disposed at second end 34, and a plurality of rings 26 is disposed between first end 32 and second end 34 and spaced along longitudinal axis A (e.g., equidistantly). In some embodiments, rings 26 are configured to wholly or in part stabilize rails 24. Rails 24 and rings 26 are configured to provide a rigid body capable of retaining catheter 10 in the fully extended orientation (i.e., without bending) when external stresses are applied to exoskeleton 22. It will be understood by one of ordinary skill in the art that exoskeleton 22 can include any number of rails 24 and rings 26 selected to provide a desired support for a chosen catheter or device to be packaged.

Rails 24 and rings 26 can be integrally formed, for example, via injection molding (e.g., single or multi-shot plastic injection molding, metal injection molding, or combination of materials). In other examples, rails 24 can be joined to rings 26 by multi-shot injection, adhesives bonding, welding, or other suitable means known in the art. Rails 24 and rings 26 can be formed of polymer compatible with sterilization gases and storage of terminally sterilized medical devices. The number, size (e.g., thickness), and spacing of rails 24 and rings 26 and material composition of rails 24 and rings 26 can be selected to provide a desired rigidity for exoskeleton 22. Generally, exoskeleton 22 can be configured to retain its cylindrical and straight shape when lifted, dropped, or otherwise moved, and when stacked with other form-fitting packaging containers 20 and other items.

All or a subset of rings 26 are configured to receive at least one of two supports, identified as supports 28A and 28B. Supports 28A and 28B are configured to stabilize the orientation and position of catheter 10 with handle 12 within form-fitting packaging container 20. One of supports 28A and 28B can be provided with each ring 26 or a subset of rings 26, as needed for stabilizing catheter 10 with handle 12. As shown in FIG. 2, all but the two rings 26 disposed at opposite ends 32 and 34 are provided with supports 28A or 28B. More rings 26 than necessary to provide rigidity to exoskeleton 22 can be provided to support catheter 10 and handle 12.

Supports 28A and 28B are aligned with rings 26 and oriented perpendicular to rails 24. Supports 28A and 28B define central openings 30A and 30B, respectively, which are configured to receive catheter 10 and handle 12. Catheter 10 is received through the smaller central opening 30B. Handle 12 is received through the larger central opening 30A. Central openings 30A and 30B can substantially match outer diameters of handle 12 and catheter 10, respectively, or can be slightly larger than the outer diameters of handle 12 and catheter 10 to allow some movement within form-fitting packaging container 20 when stress (e.g., caused by movement, compression, vibration, etc.) is applied to form-fitting packaging container 20. A small gap can be provided between supports 28A and handle 12 and supports 28B and catheter 10 to allow sterilization gases to reach all surfaces of catheter 10 and handle 12 in the packaging process. Central openings 30A and 30B can be concentric. Central openings 30A and 30B can be centered on longitudinal axis A to centrally locate catheter 10 and handle 12 in exoskeleton 22.

Form-fitting packaging container 20 can include a plurality of supports 28B to support catheter 10 and one or a plurality of supports 28B to support handle 12. The number of supports 28A and 28B can be selected to retain the position of catheter 10 with handle 12 within exoskeleton 22 and to limit movement of catheter 10 and handle 12 when external stress is applied to form-fitting packaging container 20. The number and spacing of supports 28A and 28B shown in FIG. 2 are provided for illustrative purposes only and are not intended to be limiting. It will be understood by one of ordinary skill in the art that the number of supports 28A provided to support handle 12 and the number of supports 28B provided to support catheter 10 can vary based on the configuration of catheter 10 and handle 12 (e.g., diameter, shape, weight, length, etc.). The spacing of the plurality of supports including supports 28A and 28B can be uniform or can vary. For example, it may be preferrable to include multiple supports 28A that are closely spaced to support handle 12, which is larger and heavier than catheter 10, and include multiple supports 28B spaced farther apart than supports 28A to support catheter 10.

FIG. 3 is a schematic illustration of one example of support 28A attached to a ring 26. FIG. 3 shows ring 26, support 28A, brackets 36 (herein also referred to as a pair of brackets 36), split rings 38, ends 40, retention portion 42, retention features 44, and receiving slot 46. Support 28A is a two-part support frame formed from a pair of brackets 36. The pair of brackets 36 can be symmetrical and oppositely disposed, facing the center of ring 26. Each bracket of the pair of brackets 36 includes split ring 38, retention portion 42, and receiving slot 46. Support 28A is configured to support handle 12 of catheter 10.

The pair of brackets 36 of support 28A can be provided for ease of assembly with exoskeleton 22 and handle 12. Each bracket 36 can be formed separately from exoskeleton 22. Each bracket 36 can be inserted into exoskeleton 22 between rails 24 and rings 26 and can be attached to one of the rings 26 as described further herein.

Split rings 38 can be semi-annular or arcuate. Split rings 38 are disposed adjacent to each other and together define central opening 30A. Ends 40 of adjacent split rings 38 can be disposed in contact or can be separated by a gap of sufficiently small size to prevent escape of handle 12 from opening 30A of support 28A. Adjacent split rings 38 are configured to retain the position of handle 12 within exoskeleton 22 and limit movement of handle 12 when external stress is applied to form-fitting packaging container 20.

Retention portions 42 extend radially outward from split rings 38 to ring 26. Retention portions 42 can have any shape and size suitable for supporting split rings 38 and attaching split rings 38 to rings 26. As illustrated, retention portions 42 can be solid and configured to cover a portion of the opening defined by ring 26. As shown, openings 48 can separate adjacent retention portions 42 and ring 26. In other examples, retention portions 42 can include struts, cutouts, through-holes, or other features configured to provide a desired rigidity, accommodate a retention feature, and/or accommodate sterilization gas flow therethrough.

Retention features 44 are configured to attach brackets 36 to ring 26. Retention features 44 can provide a snap-fit or press-fit connection between retention portions 42 of brackets 36 and ring 26. In one example, retention features 44 can be projections disposed on an inner perimeter of ring 26 and corresponding receiving slots 46 can be provided in retention portions 42 (FIG. 3). In other examples, retention features 44 can be projections disposed on a radially outer surface of retention portions 42 of brackets 36 and slots can be provided in ring 26, opening to the inner perimeter of ring 26. Retention features 44 and corresponding receiving slots 46 can be any shape and size suitable for attaching brackets 36 to ring 26. Generally, receiving slots 46 can be slightly smaller than retention features 44 to provide a snap-fit or press-fit connection capable of retaining attachment when external stress is applied to form-fitting packaging container 20.

As shown in FIG. 3, a single retention feature is provided for each bracket 36. In other examples, multiple retention features can be provided for each bracket 36. Single retention features 44 and corresponding receiving slots 46 of adjacent brackets 36 can be disposed directly across from each other (i.e., on opposite sides of ring 26). In other examples, a plurality of retention features 44 and corresponding receiving slots 46 can be provided for attachment of each bracket 36 and can be separated circumferentially.

Supports 28B can be substantially similar to supports 28A with split rings 38 of smaller size to define the smaller central opening 30B for positioning and retaining catheter 10. Retention portions 42 can be larger to accommodate the increased distance from split rings 38 to ring 26.

Supports 28A and 28B can be formed of a polymer compatible with sterilization gases and suitable for storage of terminally sterilized medical devices. Materials composition, thickness, size, and configuration (e.g., solid body, struts, through-holes, or openings) can be selected to provide a rigidity suitable for retaining the position of catheter 10 and handle 12 in exoskeleton 22. Supports 28A and 28B can be formed, for example, by injection molding or other processes known in the art.

In assembly, supports 28A and 28B can be inserted before catheter 10 and handle 12 is disposed in exoskeleton 22 or simultaneously with insertion of catheter 10 and handle 12. For example, one bracket 36 can be attached to each ring 26 such that a lower half of supports 28A and 28B are disposed in exoskeleton 22. Catheter 10 and handle 12 can then be inserted into exoskeleton 22 resting on the lower brackets 36 before the upper brackets 36 are inserted and attached to ring 26. In other examples, the pair of brackets 36 for each of the plurality of supports 28A and 28B can be attached to rings 26 followed by insertion of catheter 10 and handle 12 from first end 32. Catheter 10 can be inserted through first end 32 through central openings 30A and 30B, followed by insertion of handle 12.

In another example, supports 28A and 28B can be unitary members (i.e., not bracket pairs) in which ends 40 of paired brackets 36 are joined to form an annular ring defining central opening 30A or 30B. Unitary supports 28A and 28B can have substantially the same configuration as paired brackets 36. Unitary supports 28A and 28B can be inserted between adjacent rails 24 and rings 26 in assembly and can be joined to rings 26 by a press-fit. In some examples, rings 26 can have an annular flange (not shown) extending radially inward against which supports 28A and 28B can be pressed. The plurality of supports 28A and 28B can be joined to rings 26 prior to insertion of catheter 10. Catheter 10 can be inserted through central openings 30A and 30B, defined by the plurality of supports 28A and 28B, from first end 32 followed by handle 12.

In some examples end caps (not shown) can be provided at each of first end 32 and second end 34 to limit axial movement of and retain catheter 10 with handle 12 in form-fitting packaging container 20. End caps can be configured to be compatible with sterilization. Generally, sterilant gas access can be through rails 24 of exoskeleton 22. In embodiments in which voids between rails 24 do not provide sufficient access or are blocked, end caps can be perforated to allow passage of the sterilant gas.

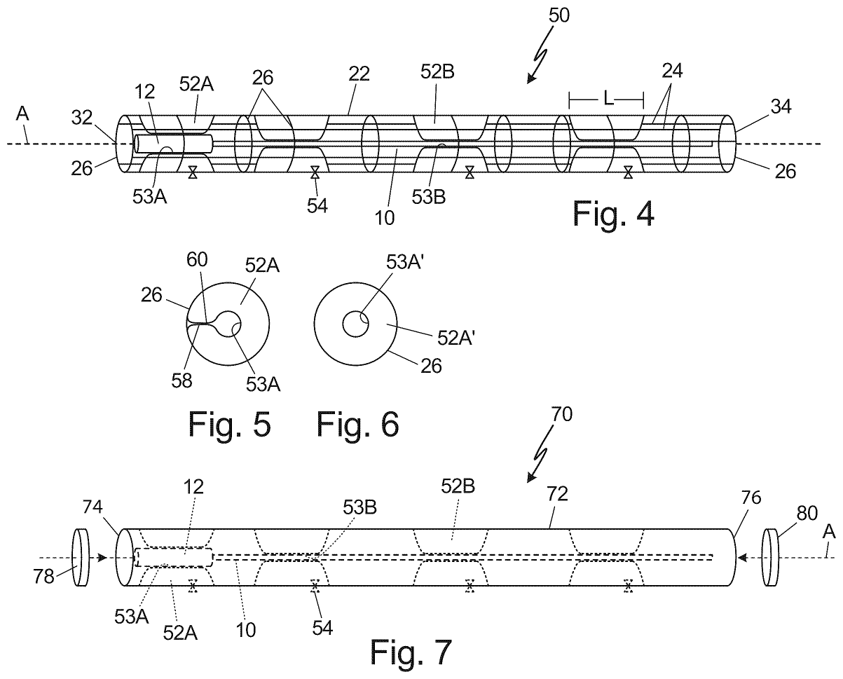

FIG. 4 is a schematic illustration of another example of a form-fitting packaging container configured for transport and storage of catheter 10. FIG. 4 shows form-fitting packaging container 50, catheter 10 with handle 12, exoskeleton 22 with rails 24 and rings 26, central openings 53A and 53B, first end 32, second end 34, supports 52A and 52B, valves 54, and longitudinal axis A. Form-fitting packaging container 50 is substantially similar to form-fitting packaging container 20 of FIG. 2. Form-fitting packaging container 50 includes exoskeleton 22, as described with respect to FIG. 2, with modified supports 52A and 52B replacing supports 28A and 28B, respectively. Exoskeleton 22, including rails 24 and rings 26 can be modified as discussed with respect to FIG. 2 to provide structural rigidity to form-fitting packaging container 50 and as may be necessary to accommodate supports 52A and 52B.

Supports 52A and 52B are inflatable bladders including valves 54 for inflating and deflating supports 52A and 52B. Supports 52A and 52B are annular bodies that define central openings 53A and 53B, respectively, which are configured to receive catheter 10 and handle 12. Catheter 10 is received through the smaller central opening 53B. Handle 12 is received through the larger central opening 53A. Central openings 53A and 53B can substantially match outer diameters of handle 12 and catheter 10, respectively, or can be slightly larger than the outer diameters of handle 12 and catheter 10 to allow some movement within form-fitting packaging container 50 when stress (e.g., caused by movement, compression, vibration, etc.) is applied to form-fitting packaging container 50. In some examples, a small gap can be provided between supports 52A and handle 12 and supports 52B and catheter 10 to allow sterilization gases to reach all surfaces of catheter 10 and handle 12 in the packaging process.

A plurality of supports 52A and 52B are longitudinally spaced about longitudinal axis A. Each support 52A and 52B can have a length L that can span across one or more rings 26. The length and number of supports 52A and 52B can be selected to retain the position of catheter 10 and handle 12 within exoskeleton 22 and limit movement of catheter 10 and handle 12 when external stress is applied to form-fitting packaging container 50. The length of each support 52A and 52B and spacing of the plurality of supports including supports 52A and 52B can be uniform or can vary. The length of each support 52A and 52B, number of supports 52A provided to support handle 12, the number of supports 52B provided to support catheter 10, and the spacing of the plurality of supports 52A and 52B can vary based on the configuration of catheter 10 and handle 12 (e.g., diameter, shape, weight, length, etc.). The lengths, number, and spacing of supports 28A and 28B shown in FIG. 4 are provided for illustrative purposes only and is not intended to be limiting.

Supports 52A and 52B can be formed of a polymer compatible with sterilization gases and storage of terminally sterilized medical devices. Supports 52A and 52B can be welded to exoskeleton 22 (i.e., rails 24 and/or rings 26), adhered to exoskeleton 22 with an adhesive compatible with sterilization gases and storage of terminally sterilized medical devices, or attached to exoskeleton 22 by a retention feature (e.g., snap-fit connection) or other suitable means. Supports 52A and 52B can be attached to an inner perimeter of exoskeleton 22 at one or more locations. In some examples, each support 52A and 52B can be attached to a full inner perimeter of a ring 26 or to all rails 24 about a circumference of exoskeleton 22. Valves 54 can be extended through exoskeleton 22 or can be disposed radially inward or radially outward of exoskeleton 22.

Supports 52A and 52B can be fully annular or can have a split ring configuration. FIG. 5 shows a split ring support 52A configured to retain handle 12 of catheter 10. Support 52A defines central opening 53A sized to receive and support handle 12. As shown in FIG. 5, support 52A extends annularly from first circumferential end 58 to second circumferential end 60. Ends 58 and 60 can be configured to contact each other upon inflation of split ring support 52A or can be separated by a gap of sufficiently small size to limit radial movement of handle 12 within exoskeleton 22.

FIG. 6 shows an annular support 52A′ (e.g., similar to a bicycle tire inner tube) configured to retain handle 12 of catheter 10. Annular support 52A′ defines central opening 53A′ sized to receive and support handle 12.

Supports 52B are substantially similar to supports 52A with modification to provide the smaller central opening 53B sized to receive and support catheter 10. Supports 52B can have a split ring or annular configuration as previously described.

Each support 52A and 52B can be partially or fully inflated before catheter 10 and handle 12 are inserted into exoskeleton 22 and/or fully inflated after catheter 10 and handle 12 are inserted into exoskeleton 22. Catheter 10 can be inserted through first end 32 through central openings 53A and 53B, followed by insertion of handle 12.

In some examples end caps (not shown) can be provided at each of first end 32 and second end 34 to limit axial movement of and retain catheter 10 with handle 12 in form-fitting packaging container 50.

Form-fitting packaging containers 20 and 50 can be disposed in a flexible sterilization enclosure (not shown) formed of a material permeable to sterilization gases and impermeable to bacteria and other contaminants. For example, form-fitting packaging containers 20 and 50 can be disposed in a synthetic fiber sheet (such as Tyvek®) pouch, sealed for sterilization and storage. Catheter 10 with handle 12 can be sterilized in form-fitting packaging containers 20 and 50 in the sterilization enclosure. Openings between rails 24 and rings 26 allow sterilization gases to infiltrate all sections of exoskeleton 22. Openings between supports 28A, 52A and handle 12 and between supports 28B, 52B and catheter 10 can allow sterilization gases to access all surfaces of catheter 10 and handle 12. All external packaging can be flexible and configured to generally conform to the shape of form-fitting packaging containers 20 and 50 in transport and storage.

FIG. 7 is a schematic illustration of yet another example of a form-fitting packaging container configured for transport and storage of catheter 10. FIG. 7 shows form-fitting packaging container 70, catheter 10 with handle 12, hollow body 72, central openings 53A and 53B, first end 74, second end 76, end caps 78 and 80, supports 52A and 52B, valves 54, and longitudinal axis A. Form-fitting packaging container 70 includes supports 52A and 52B, as described with respect to FIG. 4, in a modified hollow body 72, which replaces exoskeleton 22 of form-fitting packaging container 50.

Hollow body 72 is a solid wall cylindrical tube extending from first end 74 to second end 76. Hollow body 72 can have a rigid solid tube wall to provide structural rigidity to form-fitting packaging container 50 in transport and storage. Hollow body 72 can be formed of a polymer compatible with sterilization gases and suitable for storage of terminally sterilized medical devices. For example, hollow body 72 can be formed from high density polyethylene (HDPE).

Hollow body 72 defines the enclosure space of catheter 10. Hollow body 72 has an inner diameter greater than the largest diameter of catheter 10 (i.e., greater than the diameter of handle 12) to help isolate catheter 10 from external stresses applied to hollow body 72.

Supports 52A and 52B are inflatable bladders as described with respect to form-fitting packaging container 50 of FIG. 4. Supports 52A and 52B can be welded to the inner perimeter of hollow body 72, adhered to hollow body 72 with an adhesive compatible with sterilization gases and storage of terminally sterilized medical devices, attached to hollow body 72 by a retention feature (e.g., snap-fit connection) that extends through hollow body 72, or attached by other suitable means. Valves 54 can extend through hollow body 72 and may be used to retain a position of a respective support 52A and 52B in hollow body 72.

As previously described, the length of each support 52A and 52B and spacing of the plurality of supports including supports 52A and 52B can be uniform or can vary as appropriate for retaining a position of catheter 10 and handle 12 in hollow body 72. Supports 52A and 52B can have a split ring configuration as illustrated in FIG. 5. A small air gap can be provided between circumferential ends 58 and 60 of split ring supports 52A and 52B to allow a sterilization gas to pass through hollow body 72 from first end 32 to second end 34 (or vice versa). As previously described the gap between circumferential ends 58 and 60 can be sized to prevent escape of catheter 10 and handle 12 from central openings 53A and 53B formed by supports 52B and 52A, respectively. In some examples, the split lines of the plurality of split ring supports 52A and 52B can be circumferentially offset such that the split lines of adjacent split ring supports 52A and/or 52B do not align. This can further constrain radial movement of catheter 10 and handle 12 along the length of hollow body 72.

Catheter 10 can be inserted through central openings 53A and 53B from first end 32 followed by handle 12. Each support 52A and 52B can be partially or fully inflated before catheter 10 and handle 12 are inserted into hollow body 72 and/or fully inflated after catheter 10 and handle 12 are inserted into hollow body 72.

End caps 78 and 80 can be attached to first end 32 and second end 34. End caps 78 and 80 can be formed of a material permeable to sterilization gases and impermeable to bacteria and other contaminants. End caps 78 and 80 can be formed, for example, from synthetic fiber sheets such as Tyvek®. End caps 78 and 80 can be sealed to first end 32 and second end 34, respectively, via welding or other known methods. End caps 78 and 80 can additionally contain and restrain axial movement of catheter 10 within hollow body 72. Form-fitting packaging container 70 can be sterilized by passing sterilization gases through one of end caps 78 or 80.

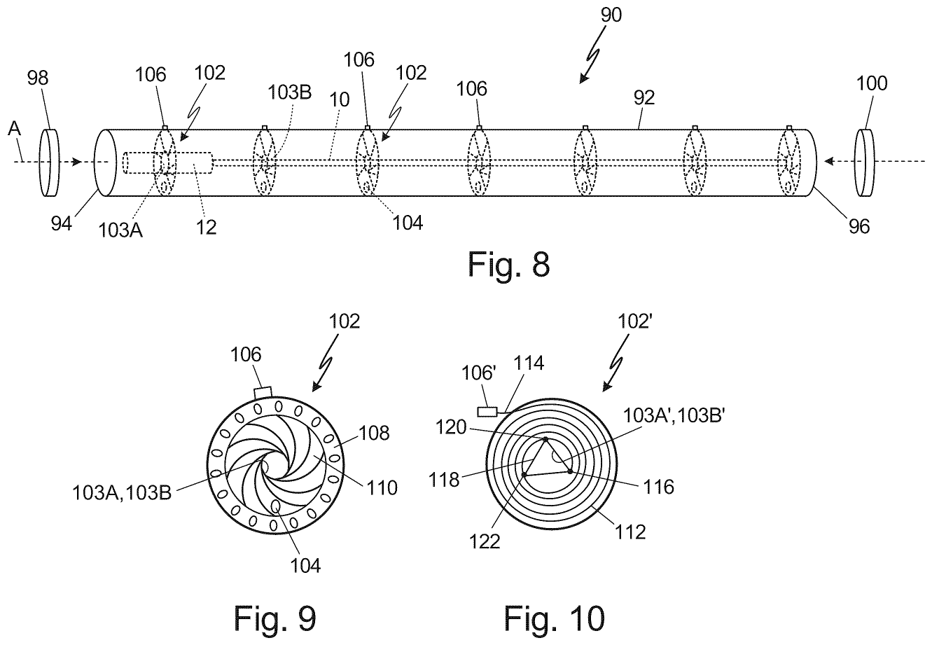

FIG. 8 is a schematic illustration of yet another example of a form-fitting packaging container configured for transport and storage of catheter 10. FIG. 8 shows form-fitting packaging container 90, catheter 10 with handle 12, hollow body 92, first end 94, second end 96, end caps 98 and 100, supports 102, central openings 103A and 103B, holes 104, actuating mechanism 106, and longitudinal axis A. Form-fitting packaging container 90 is substantially similar to form-fitting packaging container 70, illustrated in FIG. 7. Form-fitting packaging container 90 includes modified hollow body 92 to support or retain supports 102, which replace inflatable supports 52A and 52B of form-fitting packaging container 70.

Hollow body 92 can be substantially similar to hollow body 72 illustrated and described with respect to FIG. 7. Hollow body 92 can have a rigid solid tube wall to provide structural rigidity to form-fitting packaging container 90 in transport and storage. Hollow body 92 can be formed of a polymer compatible with sterilization gases and suitable for storage of terminally sterilized medical devices. For example, hollow body 92 can be formed from high density polyethylene (HDPE).

Hollow body 92 defines the enclosure space of catheter 10. Hollow body 92 has an inner diameter greater than the largest diameter of catheter 10 (i.e., greater than the diameter of handle 12) to help isolate catheter 10 from external stresses applied to hollow body 92. Hollow body 92 can include features that allow for actuation of supports 102 as described further herein.

Hollow body 92 can include a plurality of supports 102 to support catheter 10 and handle 12. The number of supports 102 can be selected to retain the position of catheter 10 with handle 12 within hollow body 92 and to limit movement of catheter 10 and handle 12 when external stress is applied to form-fitting packaging container 90. The number and spacing of supports 102 shown in FIG. 8 are provided for illustrative purposes only and are not intended to be limiting. It will be understood by one of ordinary skill in the art that the number of supports 102 provided to support catheter 10 with handle 12 can vary based on the configuration of catheter 10 and handle 12 (e.g., diameter, shape, weight, length, etc.). In some examples, it may be preferrable to include multiple supports 102 that are closely spaced to support handle 12, which is larger and heavier than catheter 10, and include multiple supports 102 spaced farther apart to support catheter 10.

Each support 102 can be configured to define central openings 103A, sized to receive and support handle 12, and 103B, sized to receive and support catheter 10. Supports 102 are disposed perpendicular to longitudinal axis A. Supports 102 can be actuated to enlarge and reduce the diameter of central openings 103A, 103B. Supports 102 can be actuated to open or increase the diameter of central openings 103A, 103B to improve the ease with which catheter 10 can be inserted into form-fitting packaging container 90. Once catheter 10 with handle 12 is disposed in form-fitting packaging container 90, supports 102 can be actuated to reduce the size of central openings 103A, 103B, such that central openings 103A, 103B close around catheter 10 and handle 12. Supports 102 can be actuated independently to control the extent to which central openings 103A, 103B open and close. Supports 102 can be actuated to contact catheter 10 and handle 12 or come into close proximity to catheter 10 and handle 12 such that central openings 103A, 103B are closely defined around catheter 10 and handle 12. Once central openings 103A, 103B are reduced to a desired diameter around catheter 10 and handle 12, supports 102 can be locked into position.

Each support 102 can include hole 104 configured to allow sterilization gases to flow through hollow body 92 from first end 94 to second end 96 (or vice versa). Holes 104 extend through supports 102. One or more holes 104 of any size suitable for allowing air flow without compromising structural support can be provided in each support 102.

In one example, supports 102 can have an iris mechanism similar to a mechanism used to adjust an aperture of a camera. FIG. 9 is a schematic illustration of support 102. FIG. 9 shows support 102, central opening 103A, 103B, hole 104, actuating mechanism 106, actuating ring 108, and blades 110. Support 102 can be configured as known in the art with blades attached to a base plate (not shown) and actuating ring 108. Actuating ring 108 can be rotated by moving actuating mechanism 106. As actuating ring 108 is rotated in a first direction, blades 110 move to open central opening 103A, 103B. As actuating ring 108 is rotated in a second, opposite, direction, blades 110 move to close opening 103A, 103B. Hole 104 can be disposed through one or more blades 110.

Actuating mechanism 106 can be disposed through a slot in hollow body 92 and can extend radially outward from hollow body 92. A slot (not shown) can be provided along a portion of the circumference of hollow body 92 to allow actuating mechanism 106 to move circumferentially to rotate actuating ring 108. Actuating mechanism 106 can be locked in an open position for assembly and locked in a retention position (i.e., desired position for retaining catheter 10 or handle 12) for transport and storage. Various locking mechanisms, including but not limited to locking push pins and holes/notches, can be used to retain the position of actuating mechanism 106 and thereby the diameter of central opening 103A, 103B. For example, a retention block (not shown) can be inserted into slots in hollow body 92 to prevent circumferential movement of actuating mechanism 106.

Supports 102 are configured to retain handle 12 or catheter 10. The degree of rotation of actuating ring 108 by actuation mechanism 106 on support 102 determines the size of central opening 103A, 103B. The degree of rotation required to reach a desired diameter of central opening 103A for handle 12 is less than the degree of rotation required to reach the desired diameter of central opening 103B for catheter 10, as catheter 10 has a smaller diameter than handle 12.

In another example, supports 102 can have a coiled spring or mainspring similar to a mechanism used to wind a watch. FIG. 10 is a schematic illustration of support 102′ using such a mechanism. FIG. 10 shows support 102′, central opening 103A′, 103B′, actuating mechanism 106′, coiled spring 112, ends 114 and 116, and central spring 118. Central spring 118 can have a triangular shape or other shape configured to expand and contract with actuation of coiled spring 112. Central spring 118 can define the shape and size of central opening 103B′. Coiled spring 112 extends from end 114 to end 116. End 114 can extend through a slot in hollow body 92 and can be attached to actuating mechanism 106 disposed outside of hollow body 92. End 116 is attached to central spring 118. Central spring 118 is additionally connected to other portions of coiled spring 112 (e.g., attachment points 120 and 122) along the length of coiled spring 112. As end 114 is pulled via actuating mechanism 106, coiled spring 112 unwinds and stretches central spring 118, enlarging central opening 103A′, 103B′. As end 114 is released or allowed to return, coiled spring 112 winds, removing stress from central spring 118 and reducing central opening 103A′, 103B′. Actuating mechanism 106′ can have any configuration suitable for pulling end 114 of coiled spring 112. Coiled spring 112 can be unwound to create a larger central opening 103A′, 103B′ during insertion of catheter 10 and handle 12. Once catheter 10 and handle 12 is positioned in hollow body 92, coiled spring 112 can be wound to reduce the size of central opening 103A′, 103B′ to a desired size to retain catheter 10 or handle 12. Coiled spring 112 can be locked in any position via a locking mechanism (not shown). Locking mechanisms can include, for example, mechanisms for securing actuating mechanism 106′ and/or end 114 and/or the portion of coiled spring 112 disposed outside of hollow body 92 to an outer surface of hollow body 92.

Supports 102′ are configured to retain handle 12 or catheter 10. The extent to which coiled spring 112 is unwound or wound by pulling or releasing actuating mechanism 106′ determines the size of central opening 103A′, 103B′. The extent to which coiled spring 112 is unwound to reach a desired size of central opening 103B′ for catheter 10 is less than the extent to which coiled spring 112 is unwound to reach a desired diameter of central opening 103A′, as catheter 10 has a smaller diameter than handle 12.

Openings between coils of coiled spring 112 can allow sterilization gases to pass through form-fitting packaging container from end 94 to end 96 (or vice versa).

Supports 102 can be inserted into hollow body 92 and secured to the hollow body 92 by mechanical fasteners, adhesives compatible with sterilization gases and storage of terminally sterilized medical devices, welding, or other suitable means.

End caps 98 and 100 can be attached to first end 94 and second end 96. End caps 98 and 100 can be formed of a material permeable to sterilization gases and impermeable to bacteria and other contaminants. End caps 98 and 100 can be formed, for example, from Tyvek®. End caps 98 and 100 can be sealed to first end 94 and second end 96, respectively, via welding or other known methods. End caps 98 and 100 can additionally contain and restrain axial movement of catheter 10 within hollow body 92. Form-fitting packaging container 90 can be sterilized by passing sterilization gases through one of end caps 98 or 100.

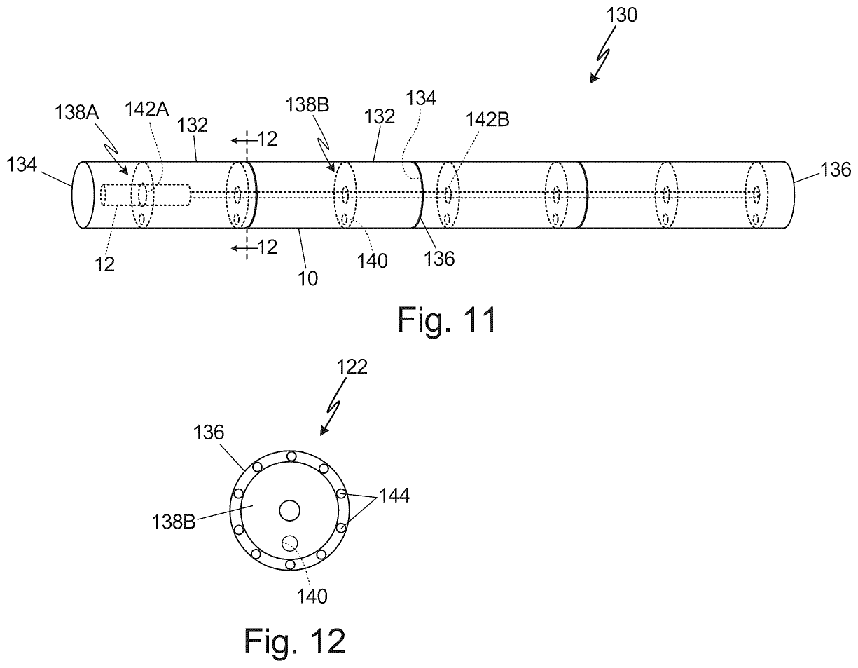

In some examples hollow bodies, including hollow bodies 72 and 92, can be formed from a plurality of hollow body segments as illustrated in FIG. 11. FIG. 11 shows form-fitting packaging container 130, hollow body segments 132, catheter 10, handle 12, ends 134 and 136, supports 138A and 138B, holes 140, central openings 142A and 142B, and longitudinal axis A. As described with the examples above, form-fitting packaging container 130 can include a plurality of supports 138B to support catheter 10 and one or a plurality of supports 138B to support handle 12. The number of supports 138A and 138B can be selected to retain the position of catheter 10 with handle 12 within form-fitting packaging container 130 and to limit movement of catheter 10 and handle 12 when external stress is applied to form-fitting packaging container 20. The number and spacing of supports 138A and 138B shown in FIG. 11 are provided for illustrative purposes only and are not intended to be limiting. It will be understood by one of ordinary skill in the art that the number of supports 138A provided to support handle 12 and the number of supports 138B provided to support catheter 10 can vary based on the configuration of catheter 10 and handle 12 and the spacing of the plurality of supports including supports 138A and 138B can be uniform or can vary as previously described.

Supports 138A and 138B can be disks having central openings 142A or 142B sized to receive and support catheter 10 and handle 12 while also permitting flow of a sterilization gas. In some examples, supports 138A and 138B can include holes 140 configured to permit flow of sterilization gas through hollow bodies 72, 92. Supports 138A and 138B are disposed perpendicular to longitudinal axis A. Supports 138A and 138B can be attached to hollow body segments 132 via a press-fit connection or other previously described means for retention or variation thereof. In other examples, supports 138A and 138B can have any configuration previously described or variation thereof. Holes 140 are configured to allow sterilizing gases to pass though form-fitting packaging container 130 from end 134 to end 136 (or vice versa).

Hollow body segments 132 are rigid cylindrical tubes having solid walls consistent with those described for hollow bodies 72 and 92. Use of hollow body segments 132 can increase the ease with which supports 138A and 138B are attached to internal walls of the hollow body 72, 92. Additionally, use of hollow body segments 132 can increase the ease with which catheter 10 can be packaged. For example, segments 132 can be added one at a time to catheter 10 such that catheter 10 does not need to be inserted blindly through the full length of the form-fitting packaging container.

FIG. 12 is a cross-sectional view taken along the 12-12 line of FIG. 11 and showing segment end 136, retention features 144, support 138B, and hole 140. Each segment 132 can include a segment end 136 with retention features 44 configured to engage end 134 of an adjacent hollow body segment 132. Retention features 144 can be, for example, projections, as illustrated in FIG. 12, configured to engage corresponding slots in end 134 of the adjacent hollow body segment 132 via a snap-fit or press-fit connection.

In other examples, hollow bodies, including hollow bodies 72 and 92, can be formed from a plurality of semi-cylindrical body segments (e.g., defining a top and bottom of the cylindrical hollow body) or a combination of semi-cylindrical body segments and cylindrical body segments. In some examples, semi-cylindrical body segments can be joined along a longitudinally extending end wall by one or more hinges.

Form-fitting packaging container 130 can have end caps (not shown) as described with respect form-fitting packaging containers 70 and 90.

The disclosed form-fitting packaging containers are configured to provide stability of a medical device during shipping and in long-term storage while reducing packaging volume.

The embodiments disclosed herein are intended to provide an explanation of the present invention and not a limitation of the invention. The present invention is not limited to the embodiments disclosed. While it may not be explicitly described, it will be understood by one of ordinary skill in the art that many of the features disclosed in one embodiment are interchangeable and combinable with features disclosed in other embodiments. Furthermore, it will be understood by one skilled in the art that various modifications and variations can be made to the invention without departing from the scope and spirit of the invention.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Any relative terms or terms of degree used herein, such as “substantially,” “essentially,” “generally,” “approximately” and the like, should be interpreted in accordance with and subject to any applicable definitions or limits expressly stated herein. In all instances, any relative terms or terms of degree used herein should be interpreted to broadly encompass any relevant disclosed embodiments as well as such ranges or variations as would be understood by a person of ordinary skill in the art in view of the entirety of the present disclosure, such as to encompass ordinary manufacturing tolerance variations, incidental alignment variations, transient alignment or shape variations induced by thermal, rotational or vibrational operational conditions, and the like. Moreover, any relative terms or terms of degree used herein should be interpreted to encompass a range that expressly includes the designated quality, characteristic, parameter, or value, without variation, as if no qualifying relative term or term of degree were utilized in the given disclosure or recitation.

### DISCUSSION OF DETAILED EMBODIMENTS

The following are non-exclusive descriptions of possible embodiments of the present invention.

In one aspect, a form-fitting packaging container for a catheter includes a hollow body extending longitudinally between a first and a second end and a plurality of supports disposed in the hollow body and spaced longitudinally between the first end and the second end. The plurality of supports define a central opening configured to receive the catheter.

The form-fitting packaging container of the preceding paragraph can optionally include, additionally and/or alternatively, any one or more of the following features, configurations, and/or additional components:

In an embodiment of the form-fitting packaging container of the preceding paragraphs, the plurality of supports can include inflatable bladders.

In an embodiment of the form-fitting packaging container of any of the preceding paragraphs, the inflatable bladders can include valves configured for inflating and venting the inflatable bladders.

In an embodiment of the form-fitting packaging container of the preceding paragraph, the inflatable bladders can have a split-ring configuration in which each inflatable bladder extends circumferentially from a first end to a second end, the first and second ends disposed adjacent to each other but separated by an air gap.

In an embodiment of the form-fitting packaging container of any of the preceding paragraphs, the hollow body can be a solid wall tube.

An embodiment of the form-fitting packaging container of the preceding paragraphs can further include end caps disposed at opposite first and second ends of the rigid tube and formed of a material permeable to sterilization gases and impermeable to bacteria.

In an embodiment of the form-fitting packaging container of any of the preceding paragraphs, the hollow body can be a cage.

In an embodiment of the form-fitting packaging container of the preceding paragraph, the cage can include rings spaced longitudinally between the first end and the second end and a plurality of longitudinally extending rails connecting the rings.

In an embodiment of the form-fitting packaging container of any of the preceding paragraphs, the plurality of supports can be integrally formed with the hollow body.

In an embodiment of the form-fitting packaging container of any of the preceding paragraphs, the plurality of supports can include retention portions extending from the hollow body to an inner body defining the central opening.

In an embodiment of the form-fitting packaging container of any of the preceding paragraphs, the hollow body can include rings spaced longitudinally between the first end and the second end and a plurality of longitudinally extending rails connecting the rings.

In an embodiment of the form-fitting packaging container of the preceding paragraph, the plurality of supports can be attached to the rings.

In an embodiment of the form-fitting packaging container of any of the preceding paragraphs, the plurality of supports can include bracket pairs, each bracket pair comprising a first bracket and a second bracket. each first and second bracket including a semi-annular body defining a portion of the central opening and a retention portion extending radially outward from the semi-annular body. The semi-annular bodies of the first and second brackets can be aligned to define the central opening.

In an embodiment of the form-fitting packaging container of the preceding paragraph, the retention portions of the first and second brackets can be attached to the rings by snap-fit connectors.

In an embodiment of the form-fitting packaging container of any of the preceding paragraphs, each retention portion can include a projection extending radially outward and wherein each ring comprises a slot corresponding to the projection and configured to receive the projection with a snap-fit connection.

In an embodiment of the form-fitting packaging container of any of the preceding paragraphs, the plurality of supports can include inflatable bladders spaced longitudinally between the first end and the second end.

In an embodiment of the form-fitting packaging container of the preceding paragraph, the inflatable bladders can include valves configured for inflating and venting the inflatable bladders.

In an embodiment of the form-fitting packaging container of any of the preceding paragraphs, the inflatable bladders can have a split-ring configuration in which each inflatable bladder extends circumferentially from a first end to a second end.

In an embodiment of the form-fitting packaging container of any of the preceding paragraphs, the inflatable bladders can be affixed to the hollow body.

In an embodiment of the form-fitting packaging container of any of the preceding paragraphs, the hollow body can be a solid wall cylindrical tube and wherein the plurality of supports comprises annular disks having a central hole defining the central opening, wherein the annular disks are spaced longitudinally in the solid wall tube between the first end and the second end and are attached to the solid wall tube by a press fit.

In an embodiment of the form-fitting packaging container of any of the preceding paragraphs, each of the plurality of annular disks can include an opening disposed between the central hole and the hollow body and configured to allow a sterilization gas to pass through the annular disk.

In an embodiment of the form-fitting packaging container of any of the preceding paragraphs, the hollow body can include a plurality of separable cylindrical segments.

In an embodiment of the form-fitting packaging container of the preceding paragraph, adjacent cylindrical segments can be attached by snap-fit connectors.

An embodiment of the form-fitting packaging container of any of the preceding paragraphs can further include end caps disposed at opposite first and second ends of the solid wall cylindrical tube and formed of a material permeable to sterilization gases and impermeable to bacteria.

In an embodiment of the form-fitting packaging container of any of the preceding paragraphs, the plurality of supports can include actuating members configured to permit the size of the central opening, defined by the support, to change.

In an embodiment of the form-fitting packaging container of any of the preceding paragraphs, the actuating members can be rotated in one direction to increase a diameter or size of the central opening and are rotated in an opposite direction to reduce the diameter or size of the central opening.

An embodiment of the form-fitting packaging container of any of the preceding paragraphs can further include a plurality of locking mechanisms configured to lock a position of the actuating members, wherein each actuating member can be locked in a different position.

In an embodiment of the form-fitting packaging container of any of the preceding paragraphs, each actuating member can include an iris mechanism comprising an actuating ring and a plurality of blades movably connected to the actuating ring.

In an embodiment of the form-fitting packaging container of any of the preceding paragraphs, each support can include a through-hole disposed between the central opening and the hollow body and configured to allow a sterilization gas to pass through the actuating member.

In an embodiment of the form-fitting packaging container of the preceding paragraphs, the through-hole can be disposed in a blade.

In an embodiment of the form-fitting packaging container of any of the preceding paragraphs, the actuating members can include a coiled spring and a central spring connected to the coiled spring and defining the central opening.

In an embodiment of the form-fitting packaging container of any of the preceding paragraphs, a first end of the coiled spring can be attached to the central spring and a second end of the coiled spring extends through a slot of the hollow body.

In an embodiment of the form-fitting packaging container of any of the preceding paragraphs, the coiled spring can be configured such that pulling the second end of the coiled spring through the hollow body enlarges the central opening and releasing the second end of the coiled spring reduces a size of the central opening.

In an embodiment of the form-fitting packaging container of any of the preceding paragraphs, the hollow body can be a solid wall tube.

An embodiment of the form-fitting packaging container of any of the preceding paragraphs can further include end caps disposed at opposite first and second ends of the solid wall cylindrical tube and formed of a material permeable to sterilization gases and impermeable to bacteria.

An embodiment of the form-fitting packaging container of any of the preceding paragraphs can further include a pouch enclosing the hollow body, the pouch formed of a material permeable to sterilization gases and impermeable to bacteria. In another aspect, method of assembling a form-fitting packaging container for a catheter includes providing a hollow body extending longitudinally, providing a plurality of supports in the hollow body, where each support defines a central opening; and inserting a catheter through the central openings of the plurality of supports.

The method of the preceding paragraph can optionally include, additionally and/or alternatively, any one or more of the following features, configurations, additional components, and/or steps:

In an embodiment of the method of the preceding paragraphs, the hollow body can be a cage comprising rings spaced longitudinally and a plurality of longitudinally extending rails connecting the rings, and further comprising inserting the plurality of supports between adjacent rails and rings.

The method of any of the preceding paragraphs can further include attaching the plurality of supports to the plurality of rings.

In an embodiment of the method of any of the preceding paragraphs, the plurality of supports the attached to the rings by snap-fit connectors.

In an embodiment of the method of any of the preceding paragraphs, the plurality of supports can include bracket pairs, each bracket pair comprising a first bracket and a second bracket, each first and second bracket including a semi-annular body defining a portion of the central opening and a retention portion extending radially outward from the semi-annular body and attached to a ring of the plurality of rings. The semi-annular bodies of the first and second brackets can be aligned to define the central opening.

In an embodiment of the method of any of the preceding paragraphs, the first bracket can be inserted and attached to the ring, followed by insertion of the catheter and insertion of the second bracket to the ring following insertion of the catheter.

In an embodiment of the method of any of the preceding paragraphs, providing a plurality of supports can include inflating bladders spaced longitudinally along the length of the hollow body.

In an embodiment of the method of any of the preceding paragraphs, providing a plurality of supports can include actuating a mechanism configured to reduce the size of the central opening.

In an embodiment of the method of any of the preceding paragraphs, the mechanism can be an iris mechanism including an actuating ring and a plurality of moveable blades configured to open and close the central opening upon rotation of the actuating ring.

In an embodiment of the method of any of the preceding paragraphs, the actuating ring can be rotated by sliding an actuation mechanism circumferentially about and outer surface of the hollow body, the actuation mechanism connected to the actuating ring through a slot in the hollow body.

In an embodiment of the method of any of the preceding paragraphs, the mechanism can be a coiled spring having a first end extending through a slot in the hollow body and a second end disposed in the hollow body and connected to a central spring, the central spring defining a shape and size of the central opening.

In an embodiment of the method of any of the preceding paragraphs, the actuating the mechanism can include pulling the second end from the hollow body to uncoil the coiled spring within the hollow body and enlarge the central opening.

In an embodiment of the method of any of the preceding paragraphs, the actuating the mechanism can include releasing the second end to allow the coiled spring to enter the hollow body and reduce the size of the central opening.

While the invention has been described with reference to an exemplary embodiment(s), it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A form-fitting packaging container for a catheter, the form-fitting packaging container comprising:
a hollow body extending longitudinally from a first end to a second end; and
a plurality of supports disposed in the hollow body and spaced longitudinally between the first end and the second end, the plurality of supports defining a central opening configured to receive the catheter.

2. The form-fitting packaging container of claim 1, wherein each of the plurality of supports comprises an inflatable bladder.

3. The form-fitting packaging container of claim 2, wherein the inflatable bladder has a split-ring configuration in which the inflatable bladder extends circumferentially from a first circumferential end to a second circumferential end, the first and second circumferential ends disposed adjacent to each other but separated by an air gap.

4. The form-fitting packaging container of claim 2 and further comprising end caps disposed at opposite first and second ends of the hollow body, wherein the hollow body is a solid wall tube and wherein the end caps are formed of a material permeable to sterilization gases and impermeable to bacteria.

5. The form-fitting packaging container of claim 2, wherein the hollow body is a cage comprising rings spaced longitudinally between the first end and the second end, and a plurality of longitudinally extending rails connecting the rings.

6. The form-fitting packaging container of claim 2, wherein the inflatable bladder is affixed to the hollow body.

7. A form-fitting packaging container for a catheter, the form-fitting packaging container comprising:
a hollow body extending longitudinally from a first end to a second end; and
a plurality of supports disposed in the hollow body and spaced longitudinally between the first end and the second end, each of the plurality of supports extending inward from the hollow body to a central opening sized to receive the catheter.

8. The form-fitting packaging container of claim 7, wherein the hollow body is a cage comprising rings spaced longitudinally between the first end and the second end and a plurality of longitudinally extending rails connecting the rings.

9. The form-fitting packaging container of claim 8, wherein each of the plurality of supports is attached to a ring.

10. The form-fitting packaging container of claim 8, wherein each of the plurality of supports comprises a bracket pairs each bracket pair comprising a first bracket and a second bracket, each first and second bracket comprising:
a semi-annular body defining a portion of the central opening; and
a retention portion extending radially outward from the semi-annular body;
wherein the semi-annular bodies of the first and second brackets are aligned to define the central opening.

11. The form-fitting packaging container of claim 10, wherein the retention portions of the first and second brackets are attached to a ring by snap-fit connectors.

12. The form-fitting packaging container of claim 11, wherein each of the retention portions comprises a projection extending radially outward and wherein the ring comprises a slot corresponding to the projection and sized to receive the projection with a snap-fit connection.

13. A form-fitting packaging container for a catheter, the form-fitting packaging container comprising:
a solid wall tube extending longitudinally from a first end to a second end; and
a plurality of supports disposed in the solid wall tube and spaced longitudinally between the first end and the second end, each of the plurality of supports extending inward from the solid wall tube to a central opening sized to receive the catheter.

14. The form-fitting packaging container of claim 13, wherein each of the plurality of supports comprises an annular disk having a central hole defining the central opening, wherein the annular disks are spaced longitudinally in the solid wall tube between the first end and the second end.

15. The form-fitting packaging container of claim 14, wherein each annular disk comprises a through hole disposed between the central opening and the solid wall tube to allow a sterilization gas to pass through the annular disk.

16. The form-fitting packaging container of claim 13, wherein the solid wall tube comprises a plurality of separable cylindrical segments attached by snap-fit connectors.

17. The form-fitting packaging container of claim 13 and further comprising end caps disposed at opposite first and second ends of the solid wall tube and formed of a material permeable to sterilization gases and impermeable to bacteria.

18. The form-fitting packaging container of claim 13, wherein each of the plurality of supports comprises:
an actuating member configured to increase a size of the central opening or decrease a size of the central opening upon actuation; and
a locking mechanism configured to lock a position of the actuating member to maintain a size of the central opening.

19. The form-fitting packaging container of claim 18, wherein each of the plurality of supports comprises a plurality of overlapping blades connected to the actuating member via an actuating ring, wherein rotation of the actuating ring by the actuating member causes the plurality of overlapping blades to move to expand or contract the central opening.

20. The form-fitting packaging container of claim 18, wherein each of the plurality of supports comprises a coiled spring and a central spring defining the central opening, wherein a first end of the coiled spring is attached to the central spring and a second end of the coiled spring extends through a slot of the solid wall tube and connects to the actuating member, wherein pulling the actuating member pulls the coiled spring through the solid tube and enlarges the central opening and releasing the actuating member returns the coiled spring to the solid tube and reduces a size of the central opening.
